(19) United States
(12) Patent Application Publication
(10) Pub. No.: US 2008/0262000 A1
(43) Pub. Date: Oct. 23, 2008

(54) 5-Alkoxyalkyl-6-alkyl-7-Aminoazolopyrimidines, Process for Their Preparation, Their Use for Controlling Harmful Fungi, and Compositions Comprising Them

(75) Inventors: Peter Schafer, Ottersheim (DE); Udo Hunger, Mannheim (DE); Maria Scherer, Landau (DE); Harald Kohle, Bobenheim (DE); Helmut Schiffer, Grossfischlingen (DE); Thomas Grote, Wachenheim (DE); Jochen Dietz, Mannheim (DE); Wassilios Grammenos, Ludwigshafen (DE); Jan Klaas Lohmann, Mannheim (DE); Bernd Muller, Frankenthal (DE); Joachim Rheinheimer, Ludwigshafen (DE); Frank Schieweck, Hessheim (DE); Anja Schwogler, Mannheim (DE)

Correspondence Address: BRINKS, HOFER, GILSON & LIONE, 2801 SLATER ROAD, SUITE 120, MORRISVILLE, NC, 27560, US

(73) Assignee: BASF Aktiengesellschaft, Ludwigshafen (DE)

(21) Appl. No.: 11/884,412

(22) PCT Filed: Feb. 14, 2006
(86) PCT No.: PCT/EP06/50922

§ 371 Date: Aug. 15, 2007

(30) Foreign Application Priority Data
Feb. 16, 2005 (DE) 10 2005 007 157.0

(51) Int. Cl. A01N 43/90; C07D 487/04; A01P 3/00

(52) U.S. Cl. 514/259.3; 544/281; 544/263; 514/259.31

(57) ABSTRACT

5-Alkoxyalkyl-6-alkyl-7-aminoazolopyrimidines of the formula I

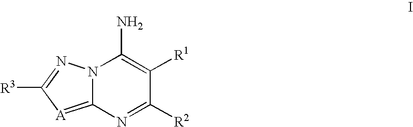

in which the substituents are defined as follows:
R¹ is alkyl, cycloalkyl, alkenyl, alkynyl, alkoxyalkyl, cyanoalkyl, and benzyloxyalkyl, where the groups in the aliphatic or aromatic moiety may be unsubstituted or may have substitution by from one to three groups R^{a}:
   R^{a} is halogen, cyano, nitro, hydroxy, cycloalkyl, alkoxy, alkylthio, and NR^{A}R^{B};
      R^{A}, R^{B} are hydrogen and alkyl;
R² is alkoxyalkyl, phenoxyalkyl, alkylthioalkyl, and phenylthioalkyl, which groups may have no substitution or may have substitution according to the description;
R³ is hydrogen and alkyl;
A is N and C—R^{A};
   processes for preparation of these compounds, compositions comprising them, and their use for controlling phytopathogenic harmful fungi.

The present invention relates to 5-alkoxyalkyl-6-alkyl-7-aminoazolopyrimidines of the formula I

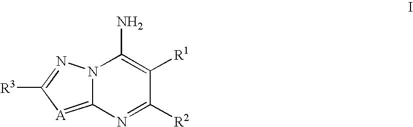

in which the substituents are defined as follows:
R¹ is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, and C₈-C₁₉-benzyloxyalkyl, where the groups in the aliphatic or aromatic moiety may be unsubstituted or may have substitution by from one to three groups R^{a}:
   R^{a} is halogen, cyano, nitro, hydroxy, C₃-C₆-cycloalkyl, C₁-C₆-alkoxy, C₁-C₆-alkylthio, and NR^{A}R^{B};
      R^{A}, R^{B} are hydrogen and C₁-C₆-alkyl;
R² is C₁-C₁₂-alkoxy-C₁-C₁₂-alkyl, phenoxy-C₁-C₁₂-alkyl, C₁-C₁₂-alkylthio-C₁-C₁₂-alkyl, and phenylthio-C₁-C₁₂-alkyl, in which groups the carbon chains can have substitution by from one to three groups R^{a}, and the phenyl rings can have substitution by from one to five substituents composed of C₁-C₆-alkyl or of a group R^{a};
R³ is hydrogen and C₁-C₆-alkyl;
A is N and C—R^{A}.

The invention also relates to processes for preparation of these compounds, compositions comprising them, and their use for controlling phytopathogenic harmful fungi.

5,6-Dialkyl-7-aminotriazolopyrimidines are proposed generally in GB 1 148 629. Individual fungicidal 5,6-dialkyl-7-aminoazolopyrimidines are disclosed in EP-A 141 317. However, their activity is unsatisfactory in many instances. Starting from this point, an object underlying the present invention is to provide compounds with improved activity and/or with a broader activity spectrum.

Accordingly; the compounds defined at the outset have been found. Processes and intermediates for their production have moreover been found, as have compositions comprising them, and also methods for controlling harmful fungi, using the compounds I.

The compounds of the formula I differ from those from the abovementioned specifications by virtue of the specific design of the substituent in the 5-position of the triazolopyrimidine skeleton.

The compounds of the formula I have increased activity against harmful fungi when compared with the known compounds.

The inventive compounds can be obtained in various ways. The inventive compounds are advantageously obtained by reacting substituted β-ketoesters of the formula II with 3-amino-1,2,4-triazole or -pyrazole of the formula III to give 7-hydroxyazolopyrimidines of the formula IV. The groups R¹ and R² in formulae II and IV are defined as for formula I, and the group R in formula II is C₁-C₄-alkyl, preference being given here to methyl, ethyl, or propyl for practical reasons.

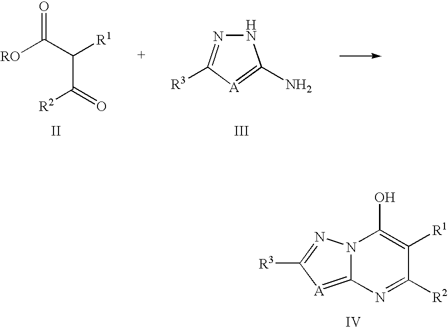

The reaction of the substituted β-ketoesters of the formula II with the aminoazoles of the formula III can be carried out in the presence or absence of solvents. It is advantageous to use solvents to which the starting materials are substantially inert and in which they are completely or to some extent soluble. Particular solvents which may be used are alcohols such as ethanol, propanols, butanols, glycols, or glycol monoethers, diethylene glycols or their monoethers, aromatic hydrocarbons such as toluene, benzene, or mesitylene, amides, such as dimethylformamide, diethylformamide, dibutylformamide, N,N-dimethylacetamide, lower alkane acids, such as formic acid, acetic acid, propionic acid, or bases, such as alkali metal hydroxides and alkaline earth metal hydroxides, alkali metal oxides and alkaline earth metal oxides, alkali metal hydrides and alkaline earth metal hydrides, alkali metal amides, alkali metal carbonates and alkaline earth metal carbonates, and also alkali metal hydrogencarbonates, organometallic compounds, in particular alkali metal alkyl compounds, alkylmagnesium halides, and also alkali metal alcoholates and alkaline earth metal alcoholates, and dimethoxymagnesium, and also organic bases, e.g. tertiary amines such as trimethylamine, triethylamine, triisopropylethylamine, tributylamine, and N-methylpiperidine, N-methylmorpholine, pyridine, substituted pyridines, such as collidine, lutidine, and 4-dimethylaminopyridine, and also bicyclic amines and mixtures of these solvents with water. Catalysts which may be used are bases, as mentioned above, or acids, such as sulfonic acid or mineral acids. The reaction is particularly preferably carried out without solvent or in chlorobenzene, xylene, dimethyl sulfoxide, N-methylpyrrolidone. Particularly preferred bases are tertiary amines, such as triisopropylamine, tributylamine, N-methylmorpholine, or N-methylpiperidine. The temperatures are from 50 to 300° C., preferably from 50 to 180° C., if preparations are carried out in solution [cf. EP-A 770 615; Adv. Het. Chem. vol., 57, pp. 81 et seq. (1993)].

The amounts used of the bases are generally catalytic amounts, but the bases may also be used in equimolar amounts, or in excess or, if appropriate, as solvents.

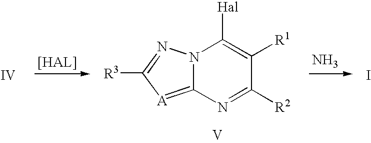

The resultant condensates of the formula IV mostly precipitate in pure form from the reaction solutions, and, after washing with the same solvent or with water and subsequent drying, are reacted with halogenating agents, in particular chlorinating or brominating agents, to give the compounds of the formula V, in which Hal: is chlorine or bromine, in particular chlorine. The reaction preferably takes place with chlorinating agents, such as phosphorus oxychloride, thionyl chloride, or sulfuryl chloride, at from 50° C. to 150° C., preferably in excess phosphorus oxytrichloride at reflux temperature. Once the excess phosphorus oxytrichloride has been evaporated, the residue is treated with iced water, if appropriate with addition of a solvent immiscible with water. The chlorination product isolated from the dried organic phase, if appropriate after evaporation of the inert solvent, is mostly very pure and is then reacted with ammonia in inert solvents at from 100° C. to 200° C. to give the 7-aminoazolo[1,5-a]pyrimidines. The reaction is preferably carried out with a from 1- to 10-molar excess of ammonia under a pressure of from 1 to 100 bar.

The novel 7-aminoazolo[1,5-a]pyrimidines are isolated as crystalline compounds via digestion in water, if appropriate after evaporation of the solvent.

The β-ketoesters of the formula II may be prepared as described in Organic Synthesis Coll. Vol. 1, p. 248, or are commercially available.

As an alternative, the novel compounds of the formula I may be obtained by reacting substituted acyl cyanides of the formula VI, in which R¹ and R² are defined as stated above, with 3-amino-1,2,4-triazole of the formula III.

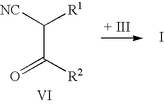

The reaction may be carried out in the presence or absence of solvents. It is advantageous to use solvents to which the starting materials are substantially inert and in which they are completely or to some extent soluble. Particular solvents which may be used are alcohols such as ethanol, propanols, butanols, glycols, or glycol monoethers, diethylene glycols or their monoethers, aromatic hydrocarbons such as toluene, benzene, or mesitylene, amides, such as dimethylformamide, diethylformamide, dibutylformamide, N,N-dimethylacetamide, lower alkane acids, such as formic acid, acetic acid, propionic acid, or bases, as mentioned above, and mixtures of these solvents with water. The reaction temperatures are from 50 to 300° C., preferably from 50 to 150° C., if operations take place in solution.

The novel 7-aminotriazolo[1,5-a]pyrimidines are isolated as crystalline compounds, if appropriate after evaporation of the solvent or dilution with water.

The substituted alkyl cyanides of the formula VI needed for preparation of the 7-aminoazolo[1,5-a]pyrimidines are to some extent known, or can be prepared by known methods from alkyl cyanides and carboxylic esters with strong bases, e.g. alkali metal hydrides, alkali metal alcoholates, alkali metal amides, or metal alkyl compounds [cf.: J. Amer. Chem. Soc. vol. 73, (1951) p. 3766].

To the extent that individual compounds I are not accessible by the routes described above, they can be prepared via derivatization of other compounds I.

The extent that isomer mixtures are produced in the synthesis, separation is not generally an essential requirement, because the individual isomers can convert into one another to some extent during procedures for use or during use (e.g. on exposure to light, to acid, or to base). Corresponding conversions can also take place after use, for example during the treatment of plants within the treated plant or within the harmful fungus to be controlled.

The definitions given for the symbols in the above formulae are collective terms which provide general representation for the following substituents:

Halogen: fluorine, chlorine, bromine, and iodine;

alkyl: saturated, straight-chain or singly or doubly branched hydrocarbon radicals having from 1 to 4 or from 5 to 12 carbon atoms, e.g. C₁-C₆-alkyl, such as methyl, ethyl, propyl, 1-methylethyl, butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-di-methylbutyl, 2,3-dimethylbutyl, 3,3-dimethylbutyl, 1-ethylbutyl, 2-ethylbutyl, 1,1,2-tri-methylpropyl, 1,2,2-trimethylpropyl, 1-ethyl-1-methylpropyl, and 1-ethyl-2-methylpropyl;

halomethyl: a methyl group in which the hydrogen atoms may have been replaced to some extent or completely by halogen atoms as mentioned above: particularly chloromethyl, bromomethyl, dichloromethyl, trichloromethyl, fluoromethyl, difluoromethyl, trifluoromethyl, chlorofluoromethyl, dichlorofluoromethyl, chlorodifluoromethyl;

cycloalkyl: mono- or bicyclic, saturated hydrocarbon groups having from 3 to 6 carbon ring members, e.g. cyclopropyl, cyclobutyl, cyclopentyl, and cyclohexyl;

alkoxyalkyl: saturated, straight-chain or singly, doubly, or triply branched hydrocarbon chain interrupted by an oxygen atom, e.g. C₅-C₁₂-alkoxyalkyl: hydrocarbon chain as described above which has from 5 to 12 carbon atoms and which can have interruptions by an oxygen atom at any desired point, e.g. propoxyethyl, butoxyethyl, pentoxyethyl, hexyloxyethyl, heptyloxyethyl, octyloxyethyl, nonyloxyethyl, 3-(3-ethylhexyloxy)ethyl, 3-(2,4,4-trimethylpentyloxy)ethyl, 3-(1-ethyl-3-methylbutoxy)ethyl, ethoxypropyl, propoxypropyl, butoxypropyl, pentoxypropyl, hexyloxypropyl, heptyloxypropyl, octyloxypropyl, nonyloxypropyl, 3-(3-ethylhexyloxy)propyl, 3-(2,4,4-tri-methylpentyloxy)propyl, 3-(1-ethyl-3-methylbutoxy)propyl, ethoxybutyl, propoxybutyl, butoxybutyl, pentoxybutyl, hexyloxybutyl, heptyloxybutyl, octyloxybutyl, nonyloxybutyl, 3-(3-ethylhexyloxy)butyl, 3-(2,4,4-trimethylpentyloxy)butyl, 3-(1-ethyl-3-methylbutoxy)butyl, methoxypentyl, ethoxypentyl, propoxypentyl, butoxypentyl, pentoxypentyl, hexyloxypentyl, heptyloxypentyl, 3-(3-methylhexyloxy)pentyl, 3-(2,4-di-methylpentyloxy)pentyl, 3-(1-ethyl-3-methylbutoxy)pentyl.

For the purposes of the present invention, the (R) ad (S) isomers and the racemates of compounds of the formula I that have chiral centers are included.

The following definitions of the substituents, in each case alone or in combination, are particularly preferred for the appropriate use of the azolopyrimidines of the formula I:

Preference is given to compounds I in which the group R¹ has at most 12 carbon atoms.

The alkyl groups in R¹ in formula I are preferably unbranched or singly, doubly, or triply branched, or multibranched groups, in particular an unbranched C₁-C₁₂-alkyl group.

Alongside this, preference is given to compounds of the formula I which have branching at the α-carbon atom in R¹. They are described by formula Ia

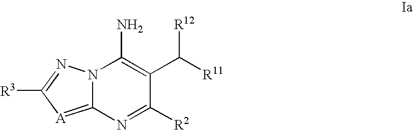

in which R¹¹ is C₃-C₁₀-alkyl or C₅-C₁₀-alkoxyalkyl, and R¹² is C₁-C₄-alkyl, in particular methyl, where R¹¹ and R¹² together have not more than 12 carbon atoms and are unsubstituted or can have substitution as R¹ in formula I, and other variables are defined as for formula I.

To the extent that R¹ is a cyano-substituted alkyl group, the cyano group is preferably on the terminal carbon atom.

To the extent that R¹ is a halo-substituted alkyl group, the halogenation is preferably present at the α- or at the ω-carbon atom.

In another preferred embodiment, R¹ is a hydroxy-substituted alkyl group.

Preference is given to compounds I in which R¹ is an unbranched or singly, doubly or triply branched, or multibranched C₅-C₁₂-alkyl group or C₅-C₁₀-alkoxypropyl group which bears no further substituents.

Particular preference is given to compounds I in which R¹ is n-pentyl, 1-methylbutyl, 2-methylbutyl, 3-methylbutyl, 2,2-dimethylpropyl, 1-ethylpropyl, n-hexyl, 1,1-dimethyl-propyl, 1,2-dimethylpropyl, 1-methylpentyl, 2-methylpentyl, 3-methylpentyl, 4-methyl-pentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl.

Preference is also given to compounds of the formula I in which R¹ is n-heptyl, 1-methylhexyl, n-octyl, 1-methylheptyl, n-nonyl, 1-methyloctyl, 3,5,5-trimethylhexyl, n-decyl, 1-methylnonyl, n-undecyl, 1-methyldecyl, n-dodecyl, and 1-methylundecyl.

In another preferred embodiment of the inventive compounds, R¹ is methoxy-n-propyl, ethoxy-n-propyl, n-propoxy-n-propyl, n-butoxy-n-propyl, n-pentyloxy-n-propyl, n-hexyl-oxypropyl, n-heptyloxy-n-propyl, n-octyloxy-n-propyl, n-nonyloxy-n-propyl or n-decyl-oxy-n-propyl.

In one preferred embodiment of the inventive compounds I, R² is C₁-C₁₂-alkoxy-C₁-C₁₂-alkyl, in particular C₁-C₁₂-alkoxymethyl.

In another preferred embodiment, R² is methoxy-C₁-C₁₂-alkyl, in particular methoxy-methyl.

In one preferred embodiment of the inventive compounds I, A is a nitrogen atom.

In another embodiment of the compounds I, A is CR⁴, in particular CH.

Preference is also given to compounds I in which R³ is hydrogen.

One particularly preferred embodiment of the inventive compounds of the formula I is provided by those of the formula I.A:

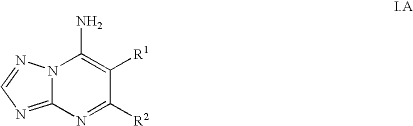

in which R¹ and R² are defined as for formula I, where R¹ is in particular C₁-C₁₂-alkyl and R² is in particular C₂-C₁₂-alkoxymethyl, preferably methoxymethyl.

With respect to their use, particular preference is given to the compounds I collated in the tables below. The groups mentioned for a substituent in the tables are moreover per se a particularly preferred embodiment of the relevant substituent, irrespective of the combination within which they have been mentioned.

### Table 1

Compounds of the formula I.A in which R¹ is a compound in each case corresponding to one line of Table A, and R² is methoxymethyl

### Table 2

Compounds of the formula I.A in which R¹ is a compound in each case corresponding to one line of Table A, and R² is ethoxymethyl

### Table 3

Compounds of the formula I.A in which R¹ is a compound in each case corresponding to one line of Table A, and R² is n-propoxymethyl

### Table 4

Compounds of the formula I.A in which R¹ is a compound in each case corresponding to one line of Table A, and R² is methoxyethyl

### Table 5

Compounds of the formula I.A in which R¹ is a compound in each case corresponding to one line of Table A, and R² is ethoxyethyl

### Table 6

Compounds of the formula I.A in which R¹ is a compound in each case corresponding to one line of Table A, and R² is n-propoxyethyl.

### Table 7

Compounds of the formula I.A in which R¹ is a compound in each case corresponding to one line of Table A, and R² is 3-methoxy-n-propyl

### Table 8

Compounds of the formula I.A in which R¹ is a compound in each case corresponding to one line of Table A, and R² is 3-ethoxy-n-propyl

### Table 9

Compounds of the formula I.A in which R¹ is a compound in each case corresponding to one line of Table A, and R² is 3-n-propoxy-n-propyl

### Table 10

Compounds of the formula I in which R¹ is a compound in each case corresponding to one line of Table A, R² is methoxymethyl, R³ is hydrogen, and A is CH

### Table 11

Compounds of the formula I in which R¹ is a compound in each case corresponding to one line of Table A, R² is ethoxymethyl, R³ is hydrogen, and A is CH

### Table 12

Compounds of the formula I in which R¹ is a compound in each case corresponding to one line of Table A, R² is n-propoxymethyl, R³ is hydrogen, and A is CH

### Table 13

Compounds of the formula I in which R¹ is a compound in each case corresponding to one line of Table A, R² is methoxyethyl, R³ is hydrogen, and A is CH

### Table 14

Compounds of the formula I in which R¹ is a compound in each case corresponding to one line of Table A, R² is ethoxyethyl, R³ is hydrogen, and A is CH

### Table 15

Compounds of the formula I in which R¹ is a compound in each case corresponding to one line of Table A, R² is n-propoxyethyl, R³ is hydrogen; and A is CH

### Table 16

Compounds of the formula I in which R¹ is a compound in each case corresponding to one line of Table A, R² is 3-methoxy-n-propyl, R³ is hydrogen, and A is CH

### Table 17

Compounds of the formula I in which R¹ is a compound in each case corresponding to one line of Table A, R² is 3-ethoxy-n-propyl, R³ is hydrogen, and A is CH

### Table 18

Compounds of the formula I in which R¹ is a compound in each case corresponding to one line of Table A, R² is 3-n-propoxy-n-propyl, R³ is hydrogen, and A is CH

### Table 19

Compounds of the formula I in which R¹ is a compound in each case corresponding to one line of Table A, R² is methoxymethyl, R³ is CH₃, and A is CH

### Table 20

Compounds of the formula I.A in which R¹ is a compound in each case corresponding to one line of Table A, R² is ethoxymethyl, R³ is CH₃, and A is CH

### Table 21

Compounds of the formula I.A in which R¹ is a compound in each case corresponding to one line of Table A, R² is n-propoxymethyl, R³ is CH₃, and A is CH

### Table 22

Compounds of the formula I.A in which R¹ is a compound in each case corresponding to one line of Table A, R² is methoxyethyl, R³ is CH₃, and A is CH

### Table 23

Compounds of the formula I.A in which R¹ is a compound in each case corresponding to one line of Table A, R² is ethoxyethyl, R³ is CH₃, and A is CH

### Table 24

Compounds of the formula I.A in which R¹ is a compound in each case corresponding to one line, of Table A, R² is n-propoxyethyl, R³ is CH₃, and A is CH

### Table 25

Compounds of the formula I.A in which R¹ is a compound in each case corresponding to one line of Table A, R² is 3-methoxy-n-propyl, R³ is CH₃, and A is CH

### Table 26

Compounds of the formula I.A in which R¹ is a compound in each case corresponding to one line of Table A, R² is 3-ethoxy-n-propyl, R³ is CH₃, and A is CH

### Table 27

Compounds of the formula I.A in which R¹ is a compound in each case corresponding to one line of Table A, R² is 3-n-propoxy-n-propyl, R³ is CH₃, and A is CH

TABLE A

| No. | R¹ |
|---|---|
| A-1 | CH₂CH₂CH₂CH₂CH₃ |
| A-2 | CH(CH₃)CH₂CH₂CH₃ |
| A-3 | CH₂CH(CH₃)CH₂CH₃ |
| A-4 | CH₂CH₂CH(CH₃)CH₃ |
| A-5 | CH₂CH₂CH(CH₃)₂ |
| A-6 | CH(CH₃)CH(CH₃)CH₃ |
| A-7 | CH(CH₃)CH(CH₃)₂ |
| A-8 | CH₂C(CH₃)₃ |
| A-9 | CH₂CH₂CH₂CH₂CH₂CH₃ |
| A-10 | CH(CH₃)CH₂CH₂CH₂CH₃ |
| A-11 | CH₂CH(CH₃)CH₂CH₂CH₃ |
| A-12 | CH₂CH₂CH(CH₃)CH₂CH₃ |
| A-13 | CH₂CH₂CH(CH₃)₂CH₂ |
| A-14 | CH₂CH₂CH₂CH(CH₃)₂ |
| A-15 | CH(CH₃)CH(CH₃)CH₂CH₃ |
| A-16 | CH(CH₃)CH₂CH(CH₃)₂ |
| A-17 | CH₂CH₂C(CH₃)₃ |
| A-18 | CH(CH₃)CH₂CH(CH₃)CH₃ |
| A-19 | CH₂CH₂CH₂CH₂CH₂CH₂CH₃ |
| A-20 | CH(CH₃)CH₂CH₂CH₂CH₂CH₃ |
| A-21 | CH₂CH(CH₃)CH₂CH₂CH₂CH₃ |
| A-22 | CH₂CH₂CH(CH₃)CH₂CH₂CH₃ |
| A-23 | CH₂CH₂CH₂CH(CH₃)CH₂CH₃ |
| A-24 | CH₂CH₂CH₂CH₂CH(CH₃)CH₃ |
| A-25 | CH₂CH₂CH₂CH₂CH(CH₃)₂ |
| A-26 | CH(CH₃)CH(CH₃)CH₂CH₂CH₃ |
| A-27 | CH₂CH(CH₃)CH(CH₃)CH₂CH₃ |
| A-28 | CH₂CH₂CH₂C(CH₃)₃ |
| A-29 | CH(CH₃)CH₂CH(CH₃)CH₂CH₃ |
| A-30 | CH₂CH(CH₃)CH(CH₃)CH₂CH₃ |
| A-31 | CH(CH₃)CH₂CH₂CH(CH₃)CH₃ |
| A-32 | CH₂CH₂CH₂CH₂CH₂CH₂CH₂CH₃ |
| A-33 | CH(CH₃)CH₂CH₂CH₂CH₂CH₂CH₃ |
| A-34 | CH₂CH(CH₃)CH₂CH₂CH₂CH₂CH₃ |
| A-35 | CH₂CH₂CH(CH₃)CH₂CH₂CH₂CH₃ |
| A-36 | CH₂CH₂CH₂CH(CH₃)CH₂CH₂CH₃ |
| A-37 | CH₂CH₂CH₂CH₂CH(CH₃)CH₂CH₃ |
| A-38 | CH₂CH₂CH₂CH₂CH₂CH(CH₃)₂ |
| A-39 | CH₂CH₂CH₂CH₂C(CH₃)₃ |
| A-40 | CH(CH₃)CH(CH₃)CH₂CH₂CH₂CH₃ |
| A-41 | CH₂CH(CH₃)CH(CH₃)CH₂CH₂CH₃ |
| A-42 | CH₂CH₂CH₂C(CH₃)₂CH₂CH₃ |
| A-43 | CH(CH₃)CH₂CH(CH₃)CH₂CH₂CH₃ |
| A-44 | CH₂CH(CH₃)CH(CH₃)CH₂CH₂CH₃ |
| A-45 | CH(CH₃)CH₂CH₂CH(CH₃)CH₂CH₃ |
| A-46 | CH(CH₃)CH₂CH₂CH₂CH(CH₃)₂ |
| A-47 | CH₂CH₂CH(CH₃)CH₂C(CH₃)₃ |
| A-48 | CH₂CH₂CH₂CH₂CH₂CH₂CH₂CH₂CH₃ |
| A-49 | CH(CH₃)CH₂CH₂CH₂CH₂CH₂CH₂CH₃ |
| A-50 | CH₂CH(CH₃)CH₂CH₂CH₂CH₂CH₂CH₃ |
| A-51 | CH₂CH₂CH(CH₃)CH₂CH₂CH₂CH₂CH₃ |
| A-52 | CH₂CH₂CH₂CH(CH₃)CH₂CH₂CH₂CH₃ |
| A-53 | CH₂CH₂CH₂CH₂CH(CH₃)CH₂CH₂CH₃ |
| A-54 | CH₂CH₂CH₂CH₂CH₂CH₂C(CH₃)₃ |
| A-55 | CH(CH₃)CH(CH₃)CH₂CH₂CH₂CH₂CH₃ |
| A-56 | CH₂CH(CH₃)CH(CH₃)CH₂CH₂CH₂CH₃ |
| A-57 | CH₂CH₂CH₂C(CH₃)₂CH₂CH₂CH₃ |
| A-58 | CH(CH₃)CH₂CH(CH₃)CH₂CH₂CH₂CH₃ |
| A-59 | CH₂CH(CH₃)CH(CH₃)CH₂CH₂CH₂CH₃ |
| A-60 | CH(CH₃)CH₂CH₂CH(CH₃)CH₂CH₂CH₃ |
| A-61 | CH(CH₃)CH₂CH₂CH₂C(CH₃)₃ |
| A-62 | CH₂CH(CH₃)CH₂CH₂CH(CH₃)₃ |
| A-63 | CH(CH₃)CH₂CH₂CH₂CH₂CH(CH₃)₂ |
| A-64 | CH₂CH(CH₃)CH₂CH₂CH₂CH(CH₃)₂ |
| A-65 | CH₂CH₂CH₂CH₂CH₂CH₂CH₂CH₂CH₂CH₃ |
| A-66 | CH(CH₃)CH₂CH₂CH₂CH₂CH₂CH₂CH₂CH₃ |
| A-67 | CH₂CH(CH₃)CH₂CH₂CH₂CH₂CH₂CH₂CH₃ |
| A-68 | CH₂CH₂CH(CH₃)CH₂CH₂CH₂CH₂CH₂CH₃ |
| A-69 | CH₂CH₂CH(CH₃)CH₂CH₂CH₂CH₂CH₂ |
| A-70 | CH₂CH₂CH₂CH(CH₃)CH₂CH₂CH₂CH₃ |
| A-71 | CH₂CH₂CH₂CH₂CH₂CH₂C(CH₃)₃ |
| A-72 | CH(CH₃)CH(CH₃)CH₂CH₂CH₂CH₂CH₂CH₃ |
| A-73 | CH₂CH(CH₃)CH(CH₃)CH₂CH₂CH₂CH₂CH₃ |
| A-74 | CH₂CH₂CH₂C(CH₃)₂CH₂CH₂CH₂CH₃ |
| A-75 | CH(CH₃)CH₂CH(CH₃)CH₂CH₂CH₂CH₂CH₃ |
| A-76 | CH₂CH(CH₃)CH(CH₃)CH₂CH₂CH₂CH₂CH₃ |
| A-77 | CH(CH₃)CH₂CH₂CH(CH₃)CH₂CH₂CH₂CH₃ |
| A-78 | CH(CH₃)CH₂CH₂CH₂CH(CH₃)CH₂CH₂CH₃ |
| A-79 | CH(CH₃)CH₂CH₂CH₂CH₂CH(CH₃)CH₂CH₃ |
| A-80 | CH(CH₃)CH₂CH₂CH₂CH₂CH₂CH(CH₃)₂ |
| A-81 | CH(CH₃)CH₂CH₂CH₂CH₂CH₂C(CH₃)CH₃ |
| A-82 | CH₂CH(CH₃)CH₂CH₂CH₂CH₂CH(CH₃)CH₃ |
| A-83 | CH(CH₃)CH₂CH₂CH₂CH₂C(CH₃)₃ |
| A-84 | CH₂CH(CH₃)CH₂CH₂CH₂C(CH₃)₃ |
| A-85 | CH₂CH₂CH₂CH₂CH₂CH₂CH₂CH₂CH₂CH₂CH₃ |
| A-86 | CH(CH₃)CH₂CH₂CH₂CH₂CH₂CH₂CH₂CH₂CH₃ |
| A-87 | CH₂CH(CH₃)CH₂CH₂CH₂CH₂CH₂CH₂CH₂CH₃ |
| A-88 | CH₂CH₂CH(CH₃)CH₂CH₂CH₂CH₂CH₂CH₂CH₃ |
| A-89 | CH₂CH₂CH₂CH(CH₃)CH₂CH₂CH₂CH₂CH₂CH₃ |
| A-90 | CH₂CH₂CH₂CH₂CH(CH₃)CH₂CH₂CH₂CH₂CH₃ |
| A-91 | CH₂CH₂CH₂CH₂CH₂CH₂CH₂C(CH₃)₃ |
| A-92 | CH(CH₃)CH(CH₃)CH₂CH₂CH₂CH₂CH₂CH₂CH₃ |
| A-93 | CH₂CH(CH₃)CH(CH₃)CH₂CH₂CH₂CH₂CH₂CH₃ |
| A-94 | CH₂CH₂CH₂C(CH₃)₂CH₂CH₂CH₂CH₂CH₃ |
| A-95 | CH(CH₃)CH₂CH(CH₃)CH₂CH₂CH₂CH₂CH₂CH₃ |
| A-96 | CH₂CH(CH₃)CH(CH₃)CH₂CH₂CH₂CH₂CH₂CH₃ |
| A-97 | CH(CH₃)CH₂CH₂CH(CH₃)CH₂CH₂CH₂CH₂CH₃ |
| A-98 | CH(CH₃)CH₂CH₂CH₂CH(CH₃)CH₂CH₂CH₂CH₃ |
| A-99 | CH(CH₃)CH₂CH₂CH₂CH₂CH(CH₃)CH₂CH₂CH₃ |
| A-100 | CH(CH₃)CH₂CH₂CH₂CH₂CH₂CH(CH₃)CH₂CH₃ |
| A-101 | CH(CH₃)CH₂CH₂CH₂CH₂CH₂CH₂CH(CH₃)₂ |
| A-102 | CH₂CH(CH₃)CH₂CH₂CH₂CH₂CH(CH₃)CH₂CH₃ |
| A-103 | CH₂CH(CH₃)CH₂CH₂CH₂CH₂CH(CH₃)CH₂CH₃ |
| A-104 | CH₂CH₂CH(CH₃)CH₂CH₂CH₂CH₂CH(CH₃)₂ |
| A-105 | CH₂CH(CH₃)CH₂CH₂CH₂CH₂C(CH₃)₃ |
| A-106 | CH₂CH₂CH₂CH₂CH₂CH₂CH₂CH₂CH₂CH₂CH₂CH₃ |
| A-107 | CH(CH₃)CH₂CH₂CH₂CH₂CH₂CH₂CH₂CH₂CH₂CH₃ |
| A-108 | CH₂CH(CH₃)CH₂CH₂CH₂CH₂CH₂CH₂CH₂CH₂CH₃ |
| A-109 | CH₂CH₂CH(CH₃)CH₂CH₂CH₂CH₂CH₂CH₂CH₂CH₃ |
| A-110 | CH₂CH₂CH₂CH(CH₃)CH₂CH₂CH₂CH₂CH₂CH₂CH₂ |
| A-111 | CH₂CH₂CH₂CH₂CH(CH₃)CH₂CH₂CH₂CH₂CH₂CH₃ |
| A-112 | CH₂CH₂CH₂CH₂CH₂CH(CH₃)CH₂CH₂CH₂CH₂CH₃ |
| A-113 | CH₂CH₂CH₂CH₂CH₂CH₂CH(CH₃)CH₂CH₂CH₂CH₃ |
| A-114 | CH₂CH₂CH₂CH₂CH₂CH₂CH₂CH(CH₃)CH₂CH₂CH₃ |
| A-115 | CH₂CH₂CH₂CH₂CH₂CH₂CH₂CH₂CH(CH₃)CH₂CH₃ |
| A-116 | CH₂CH₂CH₂CH₂CH₂CH₂CH₂CH₂CH₂CH(CH₃)₂ |
| A-117 | CH(CH₃)CH(CH₃)CH₂CH₂CH₂CH₂CH₂CH₂CH₂CH₃ |
| A-118 | CH₂CH(CH₃)CH(CH₃)CH₂CH₂CH₂CH₂CH₂CH₂CH₃ |
| A-119 | CH₂CH₂CH₂C(CH₃)₂CH₂CH₂CH₂CH₂CH₂CH₃ |
| A-120 | CH₂CH₂CH₂CH₂CH(CH₃)CH₂CH₂CH₂CH₂CH₂CH₃ |
| A-121 | CH(CH₃)CH₂CH(CH₃)CH₂CH₂CH₂CH₂CH₂CH₂CH₃ |
| A-122 | CH(CH₃)CH₂CH₂CH(CH₃)CH₂CH₂CH₂CH₂CH₂CH₃ |
| A-123 | CH(CH₃)CH₂CH₂CH₂CH(CH₃)CH₂CH₂CH₂CH₂CH₃ |
| A-124 | CH(CH₃)CH₂CH₂CH₂CH₂CH(CH₃)CH₂CH₂CH₂CH₃ |
| A-125 | CH(CH₃)CH₂CH₂CH₂CH₂CH₂CH(CH₃)CH₂CH₂CH₃ |
| A-126 | CH(CH₃)CH₂CH₂CH₂CH₂CH₂CH₂CH(CH₃)CH₂CH₃ |
| A-127 | CH₂CH(CH₃)CH₂CH₂CH₂CH₂CH(CH₃)CH₂CH₂CH₃ |
| A-128 | CH₂CH₂CH(CH₃)CH₂CH₂CH₂CH₂CH(CH₃)CH₂CH₃ |
| A-129 | CH₂CH₂CH₂CH(CH₃)CH₂CH₂CH₂CH(CH₃)CH₂CH₃ |
| A-130 | CH₂CH(CH₃)CH₂CH₂CH₂CH₂CH₂C(CH₃)₃ |
| A-131 | CH₂CH₂CH₂—O—CH₃ |
| A-132 | CH₂CH₂CH₂—O—CH₂CH₃ |
| A-133 | CH₂CH₂CH₂—O—CH₂CH₂CH₃ |
| A-134 | CH₂CH₂CH₂—O—CH₂CH₂CH₂CH₃ |
| A-135 | CH₂CH₂CH₂—O—CH₂CH₂CH₂CH₂CH₃ |
| A-136 | CH₂CH₂CH₂—O—CH₂CH₂CH₂CH₂CH₂CH₃ |
| A-137 | CH₂CH₂CH₂—O—CH₂CH₂CH₂CH₂CH₂CH₂CH₃ |
| A-138 | CH₂CH₂CH₂—O—CH₂CH₂CH₂CH₂CH₂CH₂CH₂CH₃ |
| A-139 | CH₂CH₂CH₂—O—CH₂CH₂CH₂CH₂CH₂CH₂CH₂CH₂CH₃ |
| A-140 | CH₂CH₂CH₂—O—CH(CH₃)₂ |
| A-141 | CH₂CH₂CH₂—O—C(CH₃)₃ |
| A-142 | CH₂CH₂CH₂—O—CH₂C(CH₃)₃ |
| A-143 | CH₂CH₂CH₂—O—CH(CH₃)CH₂C(CH₃)₃ |
| A-144 | CH₂CH₂CH₂—O—CH(CH₂CH₃)CH₂C(CH₃)₃ |
| A-145 | CH₂CH₂CH₂—O—CH₂CH(CH₃)CH₂CH(CH₃)₂ |
| A-146 | CH₂CH₂CH₂—O—CH₂CH(CH₂CH₃)CH₂CH₂CH₃ |
| A-147 | CH₂CH₂CH₂—O—CH₂CH₂CH(CH₃)CH₂CH(CH₃)₂ |
| A-148 | CH₂CH₂CH₂—O—CH₂CH₂CH(CH₃)CH₂C(CH₃)₃ |
| A-149 | CH₂CH₂CH₂—O—CH₂CH₂CH(CH₃)CH₂CH₂CH(CH₃)₂ |
| A-150 | CH₂CH₂CH₂—O—CH₂CH₂CH(CH₃)CH₂CH₂CH₂CH(CH₃)₂ |
| A-151 | CH₂CH₂CH₂CH₂—O—CH₃ |
| A-152 | CH₂CH₂CH₂CH₂—O—CH₂CH₃ |
| A-153 | CH₂CH₂CH₂CH₂—O—CH₂CH₂CH₃ |
| A-154 | CH₂CH₂CH₂CH₂—O—CH₂CH₂CH₂CH₃ |
| A-155 | CH₂CH₂CH₂CH₂—O—CH₂CH₂CH₂CH₂CH₃ |
| A-156 | CH₂CH₂CH₂CH₂—O—CH₂CH₂CH₂CH₂CH₂CH₃ |
| A-157 | CH₂CH₂CH₂CH₂—O—CH₂CH₂CH₂CH₂CH₂CH₂CH₃ |
| A-158 | CH₂CH₂CH₂CH₂—O—CH₂CH₂CH₂CH₂CH₂CH₂CH₂CH₃ |
| A-159 | CH₂CH₂CH₂CH₂—O—CH(CH₃)₂ |
| A-160 | CH₂CH₂CH₂CH₂—O—C(CH₃)₃ |
| A-161 | CH₂CH₂CH₂CH₂—O—CH₂C(CH₃)₃ |
| A-162 | CH₂CH₂CH₂CH₂—O—CH(CH₃)CH₂C(CH₃)₃ |
| A-163 | CH₂CH₂CH₂CH₂—O—CH(CH₂CH₃)CH₂C(CH₃)₃ |
| A-164 | CH₂CH₂CH₂CH₂—O—CH₂CH(CH₃)CH₂CH(CH₃)₂ |
| A-165 | CH₂CH₂CH₂CH₂—O—CH₂CH(CH₂CH₃)CH₂CH₂CH₃ |
| A-166 | CH₂CH₂CH₂CH₂—O—CH₂CH₂CH(CH₃)CH₂CH(CH₃)₂ |
| A-167 | CH₂CH₂CH₂CH₂—O—CH₂CH₂CH(CH₃)CH₂C(CH₃)₃ |
| A-168 | CH₂CH₂CH₂CH₂—O—CH₂CH₂CH(CH₃)CH₂CH₂CH(CH₃)₂ |
| A-169 | CH₂CH₂CH₂CH₂—O—CH₂CH₂CH(CH₃)CH₂CH₂CH₂CH(CH₃)₂ |
| A-170 | CH₂CH₂CH₂CH₂CH₂—O—CH₃ |
| A-171 | CH₂CH₂CH₂CH₂CH₂—O—CH₂CH₃ |
| A-172 | CH₂CH₂CH₂CH₂CH₂—O—CH₂CH₂CH₃ |
| A-173 | CH₂CH₂CH₂CH₂CH₂—O—CH₂CH₂CH₂CH₃ |
| A-174 | CH₂CH₂CH₂CH₂CH₂—O—CH₂CH₂CH₂CH₂CH₃ |
| A-175 | CH₂CH₂CH₂CH₂CH₂—O—CH₂CH₂CH₂CH₂CH₂CH₃ |
| A-176 | CH₂CH₂CH₂CH₂CH₂—O—CH₂CH₂CH₂CH₂CH₂CH₂CH₃ |
| A-177 | CH₂CH₂CH₂CH₂CH₂—O—CH₂CH₂CH₂CH₂CH₂CH₂CH₂CH₃ |
| A-178 | CH₂CH₂CH₂CH₂CH₂—O—CH(CH₃)₂ |
| A-179 | CH₂CH₂CH₂CH₂CH₂—O—C(CH₃)₃ |
| A-180 | CH₂CH₂CH₂CH₂CH₂—O—CH₂C(CH₃)₃ |
| A-181 | CH₂CH₂CH₂CH₂CH₂—O—CH(CH₃)CH₂C(CH₃)₃ |
| A-182 | CH₂CH₂CH₂CH₂CH₂—O—CH(CH₂CH₃)CH₂C(CH₃)₃ |
| A-183 | CH₂CH₂CH₂CH₂CH₂—O—CH₂CH(CH₃)CH₂CH(CH₃)₂ |
| A-184 | CH₂CH₂CH₂CH₂CH₂—O—CH₂CH(CH₂CH₃)CH₂CH₂CH₃ |
| A-185 | CH₂CH₂CH₂CH₂CH₂—O—CH₂CH₂CH(CH₃)CH₂CH₂CH(CH₃)₂ |
| A-186 | CH₂CH₂CH₂CH₂CH₂—O—CH₂CH₂CH(CH₃)CH₂CH(CH₃)₂ |
| A-187 | CH₂CH₂CH₂CH₂CH₂—O—CH₂CH₂CH(CH₃)CH₂C(CH₃)₃ |
| A-188 | Cyclopropyl |
| A-189 | Cyclopentyl |
| A-190 | Cyclohexyl |
| A-191 | CH═CH₂ |
| A-192 | CH₂CH═CH₂ |
| A-193 | CH═CHCH₃ |
| A-194 | C(CH₃)═CH₂ |
| A-195 | CH₂CH₂CH═CH₂ |
| A-196 | CH₂CH═CHCH₃ |
| A-197 | CH═CHCH₂CH₃ |
| A-198 | CH(CH₃)CH═CH₂ |
| A-199 | C(CH₃)═CHCH₃ |
| A-200 | CH═C(CH₃)₂ |
| A-201 | CH₂CH₂CH₂CH═CH₂ |
| A-202 | CH₂CH₂CH═CHCH₃ |
| A-203 | CH₂CH═CHCH₂CH₃ |
| A-204 | CH═CHCH₂CH₂CH₃ |
| A-205 | CH(CH₃)CH₂CH═CH₂ |
| A-206 | CH₂C(CH₃)═CHCH₃ |
| A-207 | CH₂CH═C(CH₃)₂ |
| A-208 | CH₂CH₂CH₂CH₂CH═CH₂ |
| A-209 | CH₂CH₂CH₂CH═CHCH₃ |
| A-210 | CH₂CH₂CH═CHCH₂CH₃ |
| A-211 | CH₂CH═CHCH₂CH₂CH₃ |
| A-212 | CH═CHCH₂CH₂CH₂CH₃ |
| A-213 | CH(CH₃)CH₂CH₂CH═CH₂ |
| A-214 | CH(CH₃)CH₂CH═CHCH₃ |
| A-215 | CH₂C(CH₃)═CHCH₂CH₃ |
| A-216 | CH₂CH₂CH═C(CH₃)₂ |
| A-217 | CH₂CH₂CH₂CH₂CH₂CH═CH₂ |
| A-218 | CH₂CH₂CH₂CH₂CH═CHCH₃ |
| A-219 | CH₂CH₂CH₂CH═CHCH₂CH₃ |
| A-220 | CH₂CH₂CH═CHCH₂CH₂CH₃ |
| A-221 | CH₂CH═CHCH₂CH₂CH₂CH₃ |
| A-222 | CH═CHCH₂CH₂CH₂CH₂CH₃ |
| A-223 | CH(CH₃)CH₂CH₂CH₂CH═CH₂ |
| A-224 | CH(CH₃)CH₂CH₂CH═CHCH₃ |
| A-225 | C(CH₃)═CHCH₂CH₂CH₂CH₃ |
| A-226 | CH₂CH₂CH₂CH═C(CH₃)₂ |
| A-227 | CH₂CH₂CH₂CH₂CH₂CH₂CH═CH₂ |
| A-228 | CH₂CH₂CH₂CH₂CH₂CH═CHCH₃ |
| A-229 | CH₂CH₂CH₂CH₂CH═CHCH₂CH₃ |
| A-230 | CH₂CH₂CH₂CH═CHCH₂CH₂CH₃ |
| A-231 | CH₂CH₂CH═CHCH₂CH₂CH₂CH₃ |
| A-232 | CH₂CH═CHCH₂CH₂CH₂CH₂CH₃ |
| A-233 | CH═CHCH₂CH₂CH₂CH₂CH₂CH₃ |
| A-234 | CH(CH₃)CH₂CH₂CH₂CH₂CH═CH₂ |
| A-235 | CH(CH₃)CH₂CH₂CH₂CH═CHCH₃ |
| A-236 | C(CH₃)═CHCH₂CH₂CH₂CH₂CH₃ |
| A-237 | CH₂CH₂CH₂CH₂CH═C(CH₃)₂ |
| A-238 | CH₂CH₂CH₂CH₂CH₂CH₂CH₂CH═CH₂ |
| A-239 | CH₂CH₂CH₂CH₂CH₂CH₂CH═CHCH₃ |
| A-240 | CH₂CH₂CH₂CH₂CH₂CH═CHCH₂CH₃ |
| A-241 | CH₂CH₂CH₂CH₂CH═CHCH₂CH₂CH₃ |
| A-242 | CH₂CH₂CH₂CH═CHCH₂CH₂CH₂CH₃ |
| A-243 | CH₂CH₂CH═CHCH₂CH₂CH₂CH₂CH₃ |
| A-244 | CH₂CH═CHCH₂CH₂CH₂CH₂CH₂CH₃ |
| A-245 | CH═CHCH₂CH₂CH₂CH₂CH₂CH₂CH₃ |
| A-246 | CH(CH₃)CH₂CH₂CH₂CH₂CH₂CH═CH₂ |
| A-247 | CH(CH₃)CH₂CH₂CH₂CH₂CH═CHCH₃ |
| A-248 | C(CH₃)═CHCH₂CH₂CH₂CH₂CH₂CH₃ |
| A-249 | CH₂CH₂CH₂CH₂CH₂CH═C(CH₃)₂ |
| A-250 | CH₂CH₂CH₂CH₂CH₂CH₂CH₂CH₂CH═CH₂ |
| A-251 | CH₂CH₂CH₂CH₂CH₂CH₂CH₂CH═CHCH₃ |
| A-252 | CH₂CH₂CH₂CH₂CH₂CH₂CH═CHCH₂CH₃ |
| A-253 | CH₂CH₂CH₂CH₂CH₂CH═CHCH₂CH₂CH₃ |
| A-254 | CH₂CH₂CH₂CH₂CH═CHCH₂CH₂CH₂CH₃ |
| A-255 | CH₂CH₂CH₂CH═CHCH₂CH₂CH₂CH₂CH₃ |
| A-256 | CH₂CH₂CH═CHCH₂CH₂CH₂CH₂CH₂CH₃ |
| A-257 | CH₂CH═CHCH₂CH₂CH₂CH₂CH₂CH₂CH₃ |
| A-258 | CH═CHCH₂CH₂CH₂CH₂CH₂CH₂CH₂CH₃ |
| A-259 | CH(CH₃)CH₂CH₂CH₂CH₂CH₂CH₂CH═CH₂ |
| A-260 | CH(CH₃)CH₂CH₂CH₂CH₂CH₂CH═CHCH₃ |
| A-261 | C(CH₃)═CHCH₂CH₂CH₂CH₂CH₂CH₂CH₃ |
| A-262 | CH₂CH₂CH₂CH₂CH₂CH₂CH═C(CH₃)₂ |
| A-263 | C≡CH |
| A-264 | CH₂C≡CH |
| A-265 | C≡CCH₃ |
| A-266 | CH₂CH₂C≡CH |
| A-267 | CH₂C≡CCH₃ |
| A-268 | C≡CCH₂CH₃ |
| A-269 | CH(CH₃)C≡CH |
| A-270 | CH₂CH₂CH₂C≡CH |
| A-271 | CH₂CH₂C≡CCH₃ |
| A-272 | CH₂C≡CCH₂CH₃ |
| A-273 | C≡CCH₂CH₂CH₃ |
| A-274 | CH(CH₃)CH₂C≡CH |
| A-275 | CH₂CH₂CH₂CH₂C≡CH |
| A-276 | CH₂CH₂CH₂C≡CCH₃ |
| A-277 | CH₂CH₂C≡CCH₂CH₃ |
| A-278 | CH₂C≡CCH₂CH₂CH₃ |
| A-279 | C≡CCH₂CH₂CH₂CH₃ |
| A-280 | CH(CH₃)CH₂CH₂C≡CH |
| A-281 | CH(CH₃)CH₂C≡CCH₃ |
| A-282 | CH₂CH₂CH₂CH₂CH₂C≡CH |
| A-283 | CH₂CH₂CH₂CH₂C≡CCH₃ |
| A-284 | CH₂CH₂CH₂C≡CCH₂CH₃ |
| A-285 | CH₂CH₂C≡CCH₂CH₂CH₃ |
| A-286 | CH₂C≡CCH₂CH₂CH₂CH₃ |
| A-287 | C≡CCH₂CH₂CH₂CH₂CH₃ |
| A-288 | CH(CH₃)CH₂CH₂CH₂C≡CH |
| A-289 | CH(CH₃)CH₂CH₂C≡CCH₃ |
| A-290 | CH(CH₃)CH₂C≡CCH₂CH₃ |
| A-291 | CH₂CH₂CH₂CH₂CH₂CH₂C≡CH |
| A-292 | CH₂CH₂CH₂CH₂CH₂C≡CCH₃ |
| A-293 | CH₂CH₂CH₂CH₂C≡CCH₂CH₃ |
| A-294 | CH₂CH₂CH₂C≡CCH₂CH₂CH₃ |
| A-295 | CH₂CH₂C≡CCH₂CH₂CH₂CH₃ |
| A-296 | CH₂C≡CCH₂CH₂CH₂CH₂CH₃ |
| A-297 | C≡CCH₂CH₂CH₂CH₂CH₂CH₃ |
| A-298 | CH(CH₃)CH₂CH₂CH₂CH₂C≡CH |
| A-299 | CH(CH₃)CH₂CH₂CH₂C≡CCH₃ |
| A-300 | CH₂CH₂CH₂CH₂CH₂CH₂CH₂C≡CH |
| A-301 | CH₂CH₂CH₂CH₂CH₂CH₂C≡CCH₃ |
| A-302 | CH₂CH₂CH₂CH₂CH₂C≡CCH₂CH₃ |
| A-303 | CH₂CH₂CH₂CH₂C≡CCH₂CH₂CH₃ |
| A-304 | CH₂CH₂CH₂C≡CCH₂CH₂CH₂CH₃ |
| A-305 | CH₂CH₂C≡CCH₂CH₂CH₂CH₂CH₃ |
| A-306 | CH₂C≡CCH₂CH₂CH₂CH₂CH₂CH₃ |
| A-307 | C≡CCH₂CH₂CH₂CH₂CH₂CH₂CH₃ |
| A-308 | CH(CH₃)CH₂CH₂CH₂CH₂CH₂C≡CH |
| A-309 | CH(CH₃)CH₂CH₂CH₂CH₂C≡CCH₃ |
| A-310 | CH₂CH₂CH₂CH₂CH₂CH₂CH₂CH₂C≡CH |
| A-311 | CH₂CH₂CH₂CH₂CH₂CH₂CH₂C≡CCH₃ |
| A-312 | CH₂CH₂CH₂CH₂CH₂CH₂C≡CCH₂CH₃ |
| A-313 | CH₂CH₂CH₂CH₂CH₂C≡CCH₂CH₂CH₃ |
| A-314 | CH₂CH₂CH₂CH₂C≡CCH₂CH₂CH₂CH₃ |
| A-315 | CH₂CH₂CH₂C≡CCH₂CH₂CH₂CH₂CH₃ |
| A-316 | CH₂CH₂C≡CCH₂CH₂CH₂CH₂CH₂CH₃ |
| A-317 | CH₂C≡CCH₂CH₂CH₂CH₂CH₂CH₂CH₃ |
| A-318 | C≡CCH₂CH₂CH₂CH₂CH₂CH₂CH₂CH₃ |
| A-319 | CH(CH₃)CH₂CH₂CH₂CH₂CH₂CH₂C≡CH |
| A-320 | CH(CH₃)CH₂CH₂CH₂CH₂CH₂C≡CCH₃ |
| A-321 | CH₂CH₂CN |
| A-322 | CH₂CH₂CH₂CN |
| A-323 | CH₂CH₂CH₂CH₂CN |
| A-324 | CH₂CH₂CH₂CH₂CH₂CN |
| A-325 | CH₂CH₂CH₂CH₂CH₂CH₂CN |
| A-326 | CH₂CH₂CH₂CH₂CH₂CH₂CH₂CN |
| A-327 | CH₂CH₂CH₂CH₂CH₂CH₂CH₂CH₂CN |
| A-328 | CH₂CH₂CH₂CH₂CH₂CH₂CH₂CH₂CH₂CN |
| A-329 | CH₂CH₂CH₂CH₂CH₂CH₂CH₂CH₂CH₂CH₂CN |

The compounds I are suitable as fungicides. They feature excellent activity against a broad spectrum of phytopathogenic fungi from the class of the Ascomycetes, Deuteromycetes, Oomycetes, and Basidiomycetes, in particular from the class of the Oomycetes. They have to some extent systemic activity that can be used for plant protection in the form of foliar fungicides, seed-dressing fungicides, and soil fungicides. They are particularly important for the control of a wide variety of fungi on various crop plants, such as wheat, rye, barley, oats, rice, maize, grass, banana, cotton, soybean, coffee, sugar cane, grapevines, fruit plants and ornamentals, and vegetable plants, such as cucumbers, beans, tomatoes, potatoes, and pumpkins, and also on the seed of these plants.

They are specifically suitable for control of the following plant diseases:
Alternaria species on vegetables, rapeseed, sugar cane, and fruit and rice,
Aphanomyces species on sugar cane and vegetables,
Bipolaris and Drechslera species on maize cereals, rice, and lawns,
Blumeria graminis (powdery mildew) on cereals,
Botrytis cinerea (gray mold) on strawberries, vegetables, flowers, and grapevine,
Bremia lactucae on lettuce, Cercospora species on maize, soybean, rice, and sugar cane,
Cochliobolus species on maize, cereals, rice (e.g. Cochliobolus sativus on cereals, Cochliobolus miyabeanus on rice),
Colletotricum species on soybean and cotton,
Drechslera species on cereals and maize,
Exserohilum species on maize,
Erysiphe cichoracearum and Sphaerotheca fuliginea on cucurbits,
Fusarium and Verticillium species on various plants,
Gaeumanomyces graminis on cereals
Gibberella species on cereals and rice (e.g. Gibberella fujikuroi on rice)
Grainstaining complex on rice,
Helminthosporium species on maize and rice,
Michrodochium nivale on cereals,
Mycosphaerella species on cereals, banana, and peanuts,
Phakopsara pachyrhizi and Phakopsara meibomiae on soybean,
Phomopsis species on soybean and sunflower,
Phytophthora infestans on potatoes and tomatoes,
Plasmopara viticola on grapevine,
Podosphaera leucotricha on apple,
Pseudocercosporella herpotrichoides on cereals,
Pseudoperonospora species on hops and cucurbits,
Puccinia species on cereals and maize,
Pyrenophora species on cereals,
Pyricularia oryzae, Corticium sasakii, Sarocladium oryzae, S. attenuatum, Entyloma oryzae on rice,
Pyricularia grisea on lawns and cereals,
Pythium spp. on lawns, rice, maize, cotton, rapeseed, sunflower, sugar cane, vegetables, and other plants,
Rhizoctonia species on cotton, rice, potatoes, lawns, maize, rapeseed, sugar cane, vegetables, and other plants,
Sclerotinia species on rapeseed and sunflower,
Septoria tritici and Stagonospora nodorum on wheat,
Erysiphe (syn. Uncinula) necatoron grapevine,
Setospaeria species on maize and lawns,
Sphacelotheca reilinia on maize,
Thievaliopsis species on soybean and cotton,
Tilletia species on cereals,
Ustilago species on cereals, maize, and sugar cane, and
Venturia species (scab) on apple and pear.

They are particularly suitable for control of harmful fungi from the class of the Oomycetes, such as Peronospora species, Phytophthora species, Plasmopara viticola, and Pseudoperonospora species.

The compounds I are moreover suitable for control of harmful fungi in the protection of materials (e.g. wood, paper, dispersions for paint, fibers, or textiles) and in protection of inventories. The following harmful fungi are particularly relevant in the protection of wood: ascomycetes, such as Ophiostoma spp., Ceratocystis spp., Aureobasidium pullulans, Sclerophoma spp., Chaetomium spp., Humicola spp., Petriella spp., Trichurus spp.; basidiomycetes, such as Coniophora spp., Coriolus spp., Gloeophyllum spp., Lentinus spp., Pleurotus spp., Poria spp., Serpula spp., and Tyromyces spp., deuteromycetes, such as Aspergillus spp., Cladosporium spp., Penicillium spp., Trichoderma spp., Alternaria spp., Paecilomyces spp., and zygomycetes, such as Mucor spp., and the following yeasts are also relevant in the protection of materials: Candida spp., and Saccharomyces cerevisae.

The compounds I are used by treating the fungi or the materials, seed materials, or plants to be protected from fungal infestation, or the soil, with a fungicidally effective amount of the active ingredients. The use may take place either prior to or else after infection of the materials, plants, or seed by the fungi.

The fungicidal compositions generally comprise from 0.1 to 95% by weight, preferably from 0.5 to 90% by weight, of active ingredient.

The application rates for plant-protection use depend on the desired effect and are from 0.01 to 2.0 kg of active ingredient per hectare.

Amounts of active ingredient needed for treating seed materials are generally from 1 to 1000 g/100 kg, preferably from 5 to 100 g/100 kg of seed materials.

For use in protection of materials or of inventories, the application rate of active ingredient depends on the nature of the field of use and on the desired effect. By way of example, conventional application rates in protection of materials are from 0.001 g to 2 kg, preferably from 0.005 g to 1 kg of active ingredient per cubic meter of treated materials.

The compounds of the formula I can exist in various crystalline forms, the biological activity of which can differ. They are likewise provided by the present invention.

The compounds I may be converted into the usual formulations, e.g. solutions, emulsions, suspensions, dusts, powders, pastes, and granules. The usage form depends on the particular use intended; it should always provide fine and uniform distribution of the inventive compound.

The formulations are prepared in a known manner, e.g. by extending the active ingredient with solvents and/or carrier substances, if desired with use of emulsifiers and dispersing agents. Solvents/auxiliaries which may be used for this purpose are in essence:
water, aromatic solvents (e.g. Solvesso products, xylene), paraffins (e.g. petroleum fractions), alcohols (e.g. methanol, butanol, pentanol, benzyl alcohol), ketones (e.g. cyclohexanone, gamma-butryolactone), pyrrolidones (NMP, NOP), acetates (glycol diacetate), glycols, dimethyl fatty acid amides, fatty acids, and fatty acid esters. In principle it is also possible to use solvent mixtures,
carrier substances such as ground naturally occurring minerals (e.g. kaolins, aluminas, talc, chalk) and ground synthetic minerals (e.g. fine-particle silica, silicates); emulsifiers, such as nonionic and anionic emulsifiers (e.g. polyoxyethylene fatty alcohol ethers, alkylsulfonates, and arylsulfonates), and dispersing agents, such as lignosulfites and methylcellulose.

Surfactants which may be used are the lignosulfonate of alkali metals, of alkaline earth metals, and of ammonium, naphthalenesulfonic acid, phenolsulfonic acid, dibutylnaphthalenesulfonic acid, alkylarylsulfonates, alkyl sulfates, alkylsulfonates, fatty alcohol sulfates, fatty acids, and sulfated fatty alcohol glycol ethers, and also condensates of sulfonated naphthalene and of naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ether, tributylphenyl polyglycol ether, tristearylphenyl polyglycol ether, alkylaryl polyether alcohols, alcohol- and fatty alcohol-ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors, and methylcellulose.

To produce directly sprayable solutions, emulsions, pastes, or oil dispersions, use may be made of mineral oil fractions of moderate to high boiling point, e.g. kerosene or diesel oil, and also coal tar oils and oils of vegetable or animal origin, aliphatic, cyclic, and aromatic hydrocarbons, e.g. toluene, xylene, paraffin, tetrahydronaphthalene, alkylated naphthalenes or their derivatives, methanol, ethanol, propanol, butanol, cyclohexanol, cyclohexanone, isophorone, or high-polarity solvents, e.g. dimethyl sulfoxide, N-methylpyrrolidone, or water.

Pulverulent compositions, spreadable compositions, and dustable compositions can be produced by mixing the active substances in a solid carrier or grinding these together. Granules, for example produced by encapsulation or impregnation, and homogeneous granules, may be produced via binding of the active ingredients to solid carrier substances. Examples of solid carrier substances are minerals, such as silica gels, silicates, talc, kaolin, Attaclay, limestone, lime, chalk, bole, loess, clay, dolomite, diatomaceous earth, calcium sulfate and magnesium sulfate, magnesium oxide, ground plastics, fertilizers, e.g. ammonium sulfate, ammonium phosphate, ammonium nitrate, ureas, and plant-derived products, such as cereal meal, ground tree bark, wood flour, and nutshell flour, cellulose powder, and other solid carrier substances.

The formulations generally comprise from 0.01 to 95% by weight, preferably from 0.1 to 90% by weight, of the active ingredient. The purity at which the active ingredients are used here is from 90% to 100%, preferably from 95% to 100% (by NMR spectrum).

### Examples of Formulations are: 1. Products for Dilution in Water

### A Water-Soluble Concentrates (SL, LS)

10 parts by weight of the active ingredients are dissolved using 90 parts by weight of water or of a water-soluble solvent. As an alternative, wetting agents or other auxiliaries are added. The active ingredient dissolves on dilution in water. The result is a formulation whose active ingredient content is 10% by weight.

### B Dispersible Concentrates (DC)

20 parts by weight of the active ingredients are dissolved in 70 parts by weight of cyclohexanone, with addition of 10 parts by weight of a dispersing agent, e.g. polyvinylpyrrolidone. A dispersion is produced on dilution in water. The active ingredient content is 20% by weight

### C Emulsifiable Concentrates (EC)

15 parts by weight of the active ingredients are dissolved in 75 parts by weight of xylene, with addition of Ca dodecylbenzenesulfonate and castor oil ethoxylate (in each case 5 parts by weight). An emulsion is produced on dilution in water. The active ingredient content of the formulation is 15% by weight.

### D Emulsions (EW, EO, ES)

25 parts by weight of the active ingredients are dissolved in 35 parts by weight of xylene, with addition of Ca dodecylbenzenesulfonate and castor oil ethoxylates (in each case 5 parts by weight). This mixture is added to 30 parts by weight of water by means of an emulsifying machine (Ultra-Turrax), and converted to a homogeneous emulsion. An emulsion is produced on dilution in water. The active ingredient content of the formulation is 25% by weight.

### E Suspensions (SC, OD, FS)

20 parts by weight of the active ingredients are comminuted in a stirred ball mill, with addition of 10 parts by weight of dispersing agents and wetting agents and of 70 parts by weight of water or an organic solvent, to give a fine suspension of active ingredient. A stable suspension of the active ingredient is produced on dilution in water. The active ingredient content of the formulation is 20% by weight.

### F Water-Dispersible and Water-Soluble Granules (WG, SG)

50 parts by weight of the active ingredients are finely ground, with addition of 50 parts by weight dispersing agents and wetting agents, and technical equipment (e.g. extrusion, spray tower, fluidized bed) is used to produce water-dispersible or water-soluble granules therefrom. A stable dispersion or solution of the active ingredient is produced on dilution in water. The active ingredient content of the formulation is 50% by weight.

### G Water-Dispersible and Water-Soluble Powders (WP, SP, SS, WS)

75 parts by weight of the active ingredients are milled in a rotor-stator mill, with addition of 25 parts by weight of dispersing agents and wetting agents, and also silica gel. A stable dispersion or solution of the active ingredient is produced on dilution in water. The active ingredient content of the formulation is 75% by weight.

### H Gel Formulations

20 parts by weight of the active ingredients, 10 parts by weight of dispersing agents, 1 part by weight of gelling agent, and 75 parts by weight of water or of an organic solvent are milled in a ball mill to give a fine suspension. A stable suspension with 20% by weight active ingredient content is produced on dilution with water.

### 2. Products for Direct Application

### I Dusts (DP, DS)

5 parts by weight of the active ingredients are finely ground and intimately mixed with 95 parts by weight of fine-particle kaolin. This gives a dustable product whose active ingredient content is 5% by weight.

### J Granules (GR, FG, GG, MG)

0.5 part by weight of the active ingredients is finely ground and associated with 99.5 parts by weight of carriers. Familiar processes here are extrusion, spray drying, or fluidized bed. This gives granules for direct application whose active ingredient content is 0.5% by weight.

### K ULV Solutions (UL)

10 parts by weight of the active ingredients are dissolved in 90 parts by weight of an organic solvent, e.g. xylene. This gives a product for direct application whose active ingredient content is 10% by weight.

For seed treatment it is usual to use water-soluble concentrates (LS), suspensions (FS), dusts (DS), water-dispersible and water-soluble powders (WS, SS), emulsions (ES), emulsifiable concentrates (EC), and gel formulations (GF). These formulations can be used in undiluted or preferably diluted form on the seed. Usage can precede sowing.

The active ingredients may be used as they stand, or in the form of their formulations, or in the form of usage forms prepared therefrom, e.g. in the form of directly sprayable solutions, powders, suspensions or dispersions, emulsions, oil dispersions, pastes, dusting compositions, spreading compositions, granules via spraying, misting, dusting, spreading, or pouring. The usage forms are entirely dependent on the intended uses; wherever possible they should always ensure maximum fineness of dispersion of the inventive active ingredients.

Aqueous usage forms can be prepared from emulsion concentrates, from pastes, or from wettable powders (oil dispersions) via addition of water. To prepare emulsions, pastes, or oil dispersions, the substances as they stand or dissolved in an oil or solvent may be homogenized in water by using wetting agents, tackifiers, dispersing agents, or emulsifiers. However, it is also possible to prepare concentrates composed of active substance, wetting agent, tackifier, dispersing agent, or emulsifier, and possibly solvent or oil, these concentrates being suitable for dilution with water.

The concentrations of active ingredient in the ready-to-use preparations can be varied within relatively wide ranges. They are generally from 0.0001 to 10%, preferably from 0.01 to 1%.

The active ingredients can also be used very successfully in ultralow-volume methods (ULM), and it is possible here to apply formulations with more than 95% by weight of active ingredient, or even to apply the active ingredient without additives.

Materials which may be added to the active ingredients are oils of various type, wetting agents, adjuvants, herbicides, fungicides, other pest-control compositions, and bactericides, and addition of these may, if appropriate, also be deferred until immediately prior to use (tank mix). These agents can be admixed in a ratio by weight of from 1:100 to 100:1, preferably 1:10 to 10:1, with the inventive materials. Particular adjuvants that can be used here are: organically modified polysiloxanes, e.g. Break Thru S 240®; alcohol alkoxylates, e.g. Atplus 245®, Atplus MBA 1303®, Plurafac LF 300®, and Lutensol ON 30®; EO-PO block polymerisates, e.g. Pluronic RPE 2035®, and Genapol B®; alcohol ethoxylates, e.g. Lutensol XP 80®; and sodium dioctyl sulfo-succinate, e.g. Leophen RA®.

The inventive materials can also be present in the usage form as fungicides together with other active ingredients, e.g. with herbicides, insecticides, growth regulators, fungicides, or else with fertilizers. When compounds I or compositions comprising them in the usage form as fungicides are mixed with other fungicides, the result in many instances is an enlargement of the fungicidal activity spectrum.

The following lists of fungicides with which the inventive compounds may be jointly used is intended to illustrate, but not restrict, the possibilities for combination:

### Strobilurins

azoxystrobin, dimoxystrobin, enestroburin, fluoxastrobin, kresoxim-methyl, metomino-strobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methyl-pyridin-2-ylmethoxyimino)ethyl]benzyl)carbamate, methyl 2-(ortho-(2,5-dimethylphenyl-Qxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbi phenyl-2-yl)-4-difluoro-methyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluoro-biphenyl-2-yl)-3-difluoromethyl-1-methylpyrazole-4-carboxamide, N-(2-cyano-phenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carprdpamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)-ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]pyridine;
pyrimidines: bupirimate, cyprodinil, ferimzone, fenarimol, mepanipyrim, nuarimol,
pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam; propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)pro-pionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometal compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
other: spiroxamine, cyflufenamid, cymoxanil, metrafenone.

### SYNTHESIS EXAMPLES

The specifications given in the synthesis examples below were used with appropriate modification of the starting compounds to obtain further compounds I. The compounds thus obtained are listed with physical data in the table which follows.

### Example 1

### Preparation of 3-cyano-1-methoxyundecanone

A suspension of 20.0 g (169 mmol) of potassium tert-butoxide in 120 ml of anhydrous dimethylformamide (DMF) was treated with 12.2 g (80 mmol) of decanitrile and 11.0 g (106 mmol) of methyl methoxyacetate. After 18 hours of stirring at from 20-25° C., the solvent was removed by distillation, and the residue was taken up in water and washed with cyclohexane. The aqueous phase was acidified with conc. hydrochloric acid and extracted with diethyl ether. The combined ether phases were washed with water and dried, and freed from the solvent. The residue was 8.4 g of the title compound in the form of oil, and the compound was preferably reacted without further purification.

### Example 2

### Preparation of 7-amino-5-methoxymethyl-6-octyltriazolo(1,5-a)pyrimidine

A solution of 22.0 g of the ketonitrile from Ex. 1, 8.1 g (97 mmol) of 3-amino-1,2,4-tria-zole, and 3.8 g of p-toluenesulfonic acid in 60 ml of mesitylene were heated for three hours to 180° C., whereupon some solvent was removed by distillation. The solvent was then completely removed by distillation, and the residue was taken up in dichloromethane. After washing with saturated NaHCO₃ solution and water, the organic phase was dried and freed from the solvent, and the residue was digested with diethyl ether. The residue was 15.0 g of the title compound in the form of white crystals of freezing point from 180-181° C.

TABLE I
Compounds of the formula I

| | | | | | Phys. Data |
|---|---|---|---|---|---|
| No. | R¹ | R² | R³ | A | (Fp. [° C.]) |
| I-1 | (CH₂)₇CH₃ | CH₂OCH₃ | H | N | 180-181 |
| I-2 | (CH₂)₇CH₃ | CH₂OCH₂CH₃ | H | N | 180-181 |
| I-3 | (CH₂)₃O(CH₂)₅CH₃ | CH₂OCH₃ | H | N | 133-134 |
| I-4 | (CH₂)₃O(CH₂)₇CH₃ | CH₂OCH₃ | H | N | 127-128 |
| I-5 | (CH₂)₂CH(CH₃)CH₂C(CH₃)₃ | CH₂OCH₃ | H | N | 188-189 |
| I-6 | (CH₂)₉CH₃ | (CH₂)₃S(4-CH₃—C₆H₄) | H | N | 125-127 |

### Examples of Activity Against Harmful Fungi

The fungicidal activity of the compounds of the formula I could be demonstrated via the following experiments:

The active ingredients were prepared in the form of a stock solution with 25 mg of active ingredient which was made up to 10 ml with a mixture composed of acetone and/or DMSO and of the emulsifier Uniperol® EL (wetting agent with emulsifying and dispersing action based on ethoxylated alkylphenols) in a solvent:emulsifier ratio by volume of 99:1. Water was then used to make up the volume to 100 ml. This stock solution was diluted to the active ingredient concentration stated below with the solvent/emulsifier/water mixture described.

Comparative compounds used comprise the known active ingredients A and B from EP-A 141 317, example No. 4 and 42:

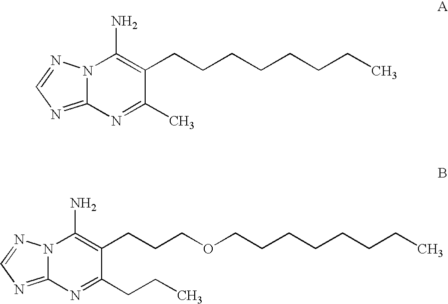

### Usage Example 1

### Activity Against Late Blight on Tomatoes by Phytophthora Infestans on Protective Treatment

Leaves of potted tomato plants were sprayed to runoff with an aqueous suspension having the concentration stated below of active ingredient. 1 day and, respectively, 7 days after application, the leaves were infected with an aqueous sporangia suspension of Phytophthora infestans. The plants were then placed in a water-vapor-saturated chamber at temperatures of from 18 to 20° C. After six days, the development of the late blight on the untreated but infected control plants was so marked that the infestation could be determined visually in %.

In the tests using 1 day of protective treatment, the plants treated with 16 ppm of the compound I-1 exhibited 15% infestation, whereas the plants treated with 16 ppm of the comparative compound A had 70%, and the untreated plants had 90% infestation. In these tests, the plants treated with 250 ppm of the compound I-4 exhibited only 1% infestation, whereas the plants treated with 250 ppm of the comparative compound B, and the untreated plants, had 90% infestation.

In another type of test using 1 day of protective treatment, the plants treated with 63 ppm of the compounds I-1, I-4, and, respectively, I-5 exhibited at most 5% infestation, whereas the untreated plants had 90% infestation.

In another type of test, with 3 days of protective treatment, the plants treated with 250 ppm of the compounds I-1 and, respectively, I-4 exhibited at most 20% infestation, whereas the plants treated with 250 ppm of the comparative compounds A and B, and also the untreated plants, had 90% infestation.

In another type of test, with 7 days of protective treatment, the plants treated with 63 ppm of the compound I-1 exhibited at most 5% infestation, whereas the untreated plants had 90% infestation.

### Usage Example 2

### Long Lasting Activity Against Peronospora on Grapevines Caused by Plasmopara Viticola on Protective Treatment

Leaves of potted vines were sprayed to runoff with an aqueous suspension having the concentration stated below of active ingredient. 1 and, respectively, 7 days after application, the undersides of the leaves were inoculated with an aqueous sporangia suspension of Plasmopara viticola. The vines were then first placed for 48 hours in a water-vapor-saturated chamber at 24° C. and were then placed for 5 days in a greenhouse at temperatures of from 20 to 30° C. After this time, the plants were again placed in a moist chamber for 16 hours to accelerate sporangiophore eruption. The extent of development of infestation on the undersides of the leaves was then determined visually.

In the tests with 1 day of protective treatment, the plants treated with 63 ppm of the compounds I-1, I-4 and, respectively, I-5 exhibited at most 3% infestation, whereas the untreated plants had 90% infestation.

In another type of test, with 7 days of protective treatment, the plants treated with 250 ppm of the compounds I-1 and, respectively, I-2 exhibited at most 5% infestation, whereas the untreated plants had 70% infestation.

1-13. (canceled)

14. A 5-alkoxyalkyl-6-alkyl-7-aminoazolopyrimidine of formula I 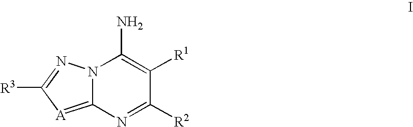
wherein the substituents are defined as follows:
R¹ is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, or C₈-C₁₉-benzyloxyalkyl, where the groups in the aliphatic or aromatic moiety are unsubstituted or are substituted with one to three groups R^{a};
R^{a} is halogen, cyano, nitro, hydroxy, C₃-C₆-cycloalkyl, C₁-C₆-alkoxy, C₁-C₆-alkylthio, or NR^{A}R^{B};
R^{A}, R^{B} are each selected from the group consisting of hydrogen and C₁-C₆-alkyl;
R² is C₁-C₁₂-alkoxy-C₁-C₁₂-alkyl, phenoxy-C₁-C₁₂-alkyl, C₁-C₁₂-alkylthio-C₁-C₁₂-alkyl, or phenylthio-C₁-C₁₂-alkyl, in which groups the carbon chains are optionally substituted with one to three groups R^{a}, and the phenyl rings are optionally substituted with one to five substituents composed of C₁-C₆-alkyl or of the group R^{a};
R³ is hydrogen or C₁-C₆-alkyl; and
A
A is N or C—R^{A}.

15. The 5-alkoxyalkyl-6-alkyl-7-aminoazolopyrimidine of formula I according to claim 14, wherein:
R² is C₁-C₁₂-alkoxy-C₁-C₁₂-alkyl or C₁-C₁₂-alkylthio-C₁-C₁₂-alkyl, in which groups the carbon chains are unsubstituted or are substituted with one to three groups R^{a}.

16. The 5-alkoxyalkyl-6-alkyl-7-aminoazolopyrimidine of formula I according to claim 14, wherein the substituents are defined as follows:
R¹ is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, C₁-C₁₂-haloalkyl, C₁-C₁₂-hydroxyalkyl, or C₈-C₁₉-benzyloxyalkyl; and
R² is C₁-C₁₂-alkoxy-C₁-C₁₂-alkyl.

17. The 5-alkoxyalkyl-6-alkyl-7-aminoazolopyrimidine of formula I according to claim 15, wherein the substituents are defined as follows:
R² is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, C₁-C₁₂-haloalkyl, C₁-C₁₂-hydroxyalkyl, or C₈-C₁₉-benzyloxyalkyl; and
R² is C₁-C₁₂-alkoxy-C₁-C₁₂-alkyl.

18. The 5-alkoxyalkyl-6-alkyl-7-aminoazolopyrimidine of formula I according to claim 14, wherein R¹ is an unsubstituted unbranched or singly, doubly, or triply branched C₁-C₁₂-alkyl chain, C₂-C₁₂-cyanoalkyl chain, C₁-C₁₂-haloalkyl chain, or C₁-C₁₂-hydroxyalkyl chain.

19. The 5-alkoxyalkyl-6-alkyl-7-aminoazolopyrimidine of formula I according to claim 14, wherein R² is C₁-C₁₂-alkoxymethyl.

20. The 5-alkoxyalkyl-6-alkyl-7-aminoazolopyrimidine of formula I according to claim 14, which corresponds to the formula I.A 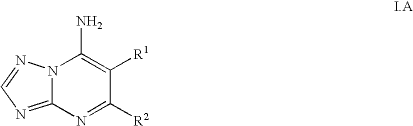
wherein
R¹ is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, or C₈-C₁₉-benzyloxyalkyl, where the groups in the aliphatic or aromatic moiety are unsubstituted or are substituted with one to three groups R^{a};
R^{a} is halogen, cyano, nitro, hydroxy, C₃-C₆-cycloalkyl, C₁-C₆-alkoxy, C₁-C₆-alkylthio, or NR^{A}R^{B};
R^{A}, R^{B} are each selected from the group consisting of hydrogen and C₁-C₆-alkyl; and
R² is C₁-C₁₂-alkoxy-C₁-C₁₂-alkyl, phenoxy-C₁-C₁₂-alkyl, C₁-C₁₂-alkylthio-C₁-C₁₂-alkyl, or phenylthio-C₁-C₁₂-alkyl, in which groups the carbon chains are optionally substituted with one to three groups R^{a}, and the phenyl rings are optionally substituted with one to five substituents composed of C₁-C₆-alkyl or of the group R^{a}.

21. A process for preparation of compounds of formula I: 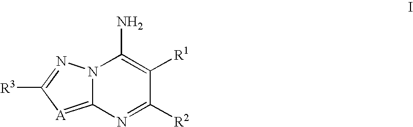
wherein the substituents are defined as follows:
R¹ is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, or C₈-C₁₉-benzyloxyalkyl, where the groups in the aliphatic or aromatic moiety are unsubstituted or are substituted with one to three groups R^{a};
R^{a} is halogen, cyano, nitro, hydroxy, C₃-C₆-cycloalkyl, C₁-C₆-alkoxy, C₁-C₆-alkylthio, or NR^{A}R^{B};
R^{A}, R^{B} are each selected from the group consisting of hydrogen and C₁-C₆-alkyl;
R² is C₁-C₁₂-alkoxy-C₁-C₁₂-alkyl, phenoxy-C₁-C₁₂-alkyl, C₁-C₁₂-alkylthio-C₁-C₁₂-alkyl, or phenylthio-C₁-C₁₂-alkyl, in which groups the carbon chains are optionally substituted with one to three groups R^{a}, and the phenyl rings are optionally substituted with one to five substituents composed of C₁-C₆-alkyl or of the group R^{a};
R³ is hydrogen or C₁-C₆-alkyl; and
A is N or C—R^{A};
comprising reacting β-ketoesters of the formula II, 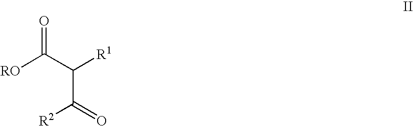
wherein R is C₁-C₄-alkyl, with 3-amino-1,2,4-triazole or -pyrazole of the formula III 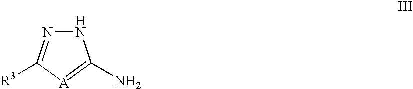
to give 7-hydroxyazolopyrimidines of the formula IV 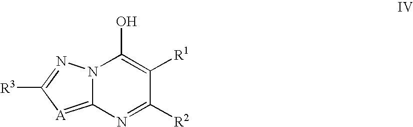
which are halogenated to give compounds of the formula V, 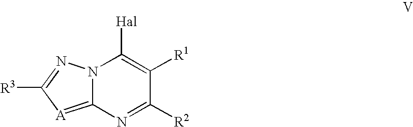
where Hal is chlorine or bromine, and reacting V with ammonia.

22. The process of claim 21, wherein the compound of the formula IV and the compound of the formula V, R¹ is an unbranched or singly, doubly, triply or multiply branched C₅-C₁₂-alkyl group or C₅-C₁₀-alkoxypropyl group.

23. A process for preparation of compounds of the formula I: 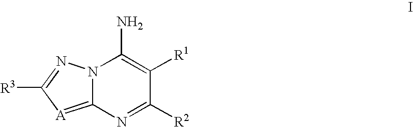
wherein the substituents are defined as follows:
R¹ is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, or C₈-C₁₉-benzyloxyalkyl, where the groups in the aliphatic or aromatic moiety are unsubstituted or are optionally substituted with one to three groups R^{a};
R^{a} is halogen, cyano, nitro, hydroxy, C₃-C₆-cycloalkyl, C₁-C₆-alkoxy, C₁-C₆-alkylthio, or NR^{A}R^{B};
R^{A}, R^{B} are each selected from the group consisting of hydrogen and C₁-C₆-alkyl;
R² is C₁-C₁₂-alkoxy-C₁-C₁₂-alkyl, phenoxy-C₁-C₁₂-alkyl, C₁-C₁₂-alkylthio-C₁-C₁₂-alkyl, or phenylthio-C₁-C₁₂-alkyl, in which groups the carbon chains are optionally substituted with one to three groups R^{a}, and the phenyl rings are optionally substituted with one to five substituents composed of C₁-C₆-alkyl or of the group R^{a};
R³ is hydrogen or C₁-C₆-alkyl; and
A is N or C—R^{A},
comprising reacting acylcyanides of the formula VI, 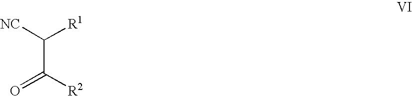
with 3-amino-1,2,4-triazole or -pyrazole of the formula III:

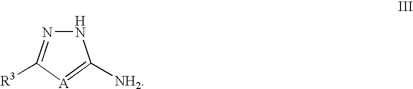

24. A composition comprising a solid or liquid carrier and a compound of formula I: 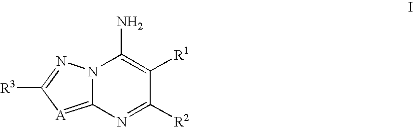
wherein the substituents are defined as follows:
R¹ is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, or C₈-C₁₉-benzyloxyalkyl, where the groups in the aliphatic or aromatic moiety are unsubstituted or are optionally substituted with one to three groups R^{a};
R^{a} is halogen, cyano, nitro, hydroxy, C₃-C₆-cycloalkyl, C₁-C₆-alkoxy, C₁-C₆-alkylthio, or NR^{A}R^{B};
R^{A}, R^{B} are each selected from the group consisting of hydrogen and C₁-C₆-alkyl;
R² is C₁-C₁₂-alkoxy-C₁-C₁₂-alkyl, phenoxy-C₁-C₁₂-alkyl, C₁-C₁₂-alkylthio-C₁-C₁₂-alkyl, or phenylthio-C₁-C₁₂-alkyl, in which groups the carbon chains are optionally substituted with one to three groups R^{a}, and the phenyl rings are optionally substituted with one to five substituents composed of C₁-C₆-alkyl or of the group R^{a};
R³ is hydrogen or C₁-C₆-alkyl; and
A is Nor C—R^{A}.

25. The composition according to claim 24, wherein
R² is C₁-C₁₂-alkoxy-C₁-C₁₂-alkyl or C₁-C₁₂-alkylthio-C₁-C₁₂-alkyl, in which groups the carbon chains are unsubstituted or are substituted with one to three groups R^{a}.

26. The composition according to claim 24, comprising a further active ingredient.

27. A seed material comprising, per 100 kg of seed, an amount of from 1 to 1000 g of a compound of formula I: 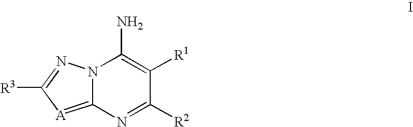
wherein the substituents are defined as follows:
R¹ is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, or C₈-C₁₉-benzyloxyalkyl, where the groups in the aliphatic or aromatic moiety are unsubstituted or are optionally substituted with one to three groups R^{a};
R^{a} is halogen, cyano, nitro, hydroxy, C₃-C₆-cycloalkyl, C₁-C₆-alkoxy, C₁-C₆-alkylthio, or NR^{A}R^{B};
R^{A}, R^{B} are each selected from the group consisting of hydrogen and C₁-C₆-alkyl;
R² is C₁-C₁₂-alkoxy-C₁-C₁₂-alkyl, phenoxy-C₁-C₁₂-alkyl, C₁-C₁₂-alkylthio-C₁-C₁₂-alkyl, or phenylthio-C₁-C₁₂-alkyl, in which groups the carbon chains are optionally substituted with one to three groups R^{a}, and the phenyl rings are optionally substituted with one to five substituents composed of C₁-C₆-alkyl or of the group R^{a};
R³ is hydrogen or C₁-C₆-alkyl; and
A is Nor C—R^{A}.

28. The seed material according to claim 27, wherein
R² is C₁-C₁₂-alkoxy-C₁-C₁₂-alkyl or C₁-C₁₂-alkylthio-C₁-C₁₂-alkyl, in which groups the carbon chains are unsubstituted or are substituted with one to three groups R^{a}.

29. A method for controlling phytopathogenic harmful fungi, which comprises treating the fungi, or the materials to be protected from fungal infestation, or plants, or the soil, or seed materials, with an effective amount of a compound of formula I: 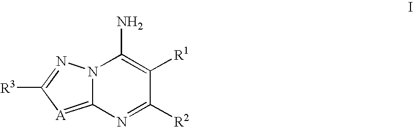
wherein the substituents are defined as follows:
R¹ is C₁-C₁₂-alkyl, C₃-C₆-cycloalkyl, C₂-C₁₂-alkenyl, C₂-C₁₂-alkynyl, C₂-C₁₂-alkoxyalkyl, C₂-C₁₂-cyanoalkyl, or C₈-C₁₉-benzyloxyalkyl, where the groups in the aliphatic or aromatic moiety are unsubstituted or are optionally substituted with one to three groups R^{a};
R^{a} is halogen, cyano, nitro, hydroxy, C₃-C₆-cycloalkyl, C₁-C₆-alkoxy, C₁-C₆-alkylthio, or NR^{A}R^{B};
R^{A}, R^{B} are each selected from the group consisting of hydrogen and C₁-C₆-alkyl; and
R² is C₁-C₁₂-alkoxy-C₁-C₁₂-alkyl, phenoxy-C₁-C₁₂-alkyl, C₁-C₁₂-alkylthio-C₁-C₁₂-alkyl, or phenylthio-C₁-C₁₂-alkyl, in which groups the carbon chains are optionally substituted with one to three groups R^{a}, and the phenyl rings are optionally substituted with one to five substituents composed of C₁-C₆-alkyl or of the group R^{a};
R³ is hydrogen or C₁-C₆-alkyl; and
A is N or C—R^{A}.

30. The method according to claim 29, wherein
R² is C₁-C₁₂-alkoxy-C₁-C₁₂-alkyl and C₁-C₁₂-alkylthio-C₁-C₁₂-alkyl, in which groups the carbon chains are unsubstituted or are substituted with one to three groups R^{a}.
